(19) United States
(12) Patent Application Publication
(10) Pub. No.: US 2014/0213949 A1
(43) Pub. Date: Jul. 31, 2014

(54) Orthopedic Brace with Sloping Paddle Members

(71) Applicant: Corflex, Inc., Manchester, NH (US)

(72) Inventors: Steven Santaniello, Cranston, RI (US); Benjamin Glace, Dunbarton, NH (US)

(21) Appl. No.: 14/169,582

(22) Filed: Jan. 31, 2014

Related U.S. Application Data
(60) Provisional application No. 61/758,967, filed on Jan. 31, 2013.

(51) Int. Cl. A61F 5/01

(52) U.S. Cl. CPC A61F 5/0102
USPC 602/16

(57) ABSTRACT

The flexible, sloping paddle members of an orthopedic brace reduce brace migration. The flexible, sloping paddle members comprise males and/or relief cuts in the paddle members to reduce brace migration and contribute to a better fitting brace. Each paddle member is centrally connected to a connector member and can be flexed around a patient's limb. The face of the paddle member slopes so that when the paddle member is flexed to surround a patient's limb, the face forms a cavity in the shape of a conical section, which is complementary to a patients limb.

### CROSS REFERENCE TO RELATED APPLICATIONS

This application claims the benefit of U.S. Provisional Application No. 61/758,967, filed Jan. 31, 2013, the content of which is incorporated by reference herein in its entirety.

### FIELD OF THE INVENTION

The present invention relates to orthopedic braces, and more particularly, to orthopedic braces with flexible, sloping paddle members.

### BACKGROUND OF THE INVENTION

There are many forms of orthoses, or devices used externally to modify the structure and/or function of the skeletal and/or neuromuscular systems of the body. For example, there are orthoses that are applied to the neck, to the spine, to the upper limbs, and to the lower limbs. Additionally, there are many different purposes for using orthoses ranging from rehabilitative to prophylactic. Rehabilitative braces are typically used to limit the movement of a portion of the body following an injury or a surgery.

Historically a major concern in orthopedic bracing is the problem of “brace migration.” Brace migration occurs when an applied orthotic brace migrates down the limb. Brace migration has many drawbacks including patient discomfort and improper brace alignment. A poor fitting brace decreases the brace's therapeutic properties and over time can reduce user compliance. Despite the widespread problem of brace migration, minimal progress has been made in this field.

The flexible, sloping paddle members of the orthopedic braces of the present invention reduce brace migration. In addition, the angles and relief cuts in the paddle members of the orthotic brace of the present invention reduce brace migration and contribute to a better fitting brace.

### SUMMARY OF THE INVENTION

The present invention is a system comprising an adjustable orthotic brace that has a pair of telescoping struts extending off a hinge and a plurality of support members that are located along the pair of telescoping struts. Each support member has a connector member and a paddle member, such that the paddle member is centrally connected to the connector member and can be flexed around a patient's limb.

The face of the paddle member slopes so that when the paddle member is flexed to surround a patient's limb, the face forms a cavity in the shape of a conical section. The paddle member can be located along the limb so that the conical section of the cavity is complementary to a conical section of the patient's limb, reducing brace migration.

In addition, the paddle members of another embodiment of the orthotic brace of the present invention have angles and relief cuts that allow them to flex more easily and conform better to a patient's limb.

One aspect of the present invention is an adjustable orthotic brace, comprising: at least one telescoping strut; and a plurality of support members located along the telescoping strut, wherein each of the support members has a connector member and a paddle member, wherein the paddle member is centrally connected to the connector member and has a pair of wing members with faces that form a conical cavity section when the paddle member is flexed to surround a patient's limb.

One embodiment of the present invention is wherein the conical cavity section formed by the faces of the pair of wing members is complementary to a conical section of a patient's limb.

One embodiment of the present invention is wherein each of the paddle members further comprises a first angle and a second angle that define the orientation of the wing members in relation to the remainder of the paddle member allowing greater flexibility of the paddle member.

One embodiment of the present invention is wherein each of the paddle members further comprises relief cuts that allow the paddle member to flex more easily to conform better to a patient's limb.

Another aspect of the present invention is a method for reducing orthotic brace migration, comprising: providing an adjustable orthotic brace with at least one telescoping strut; providing a plurality of support members located along the telescoping strut, wherein each of the support members has a connector member and a paddle member and the paddle member has a central axis and a pair of wing members; connecting each of the paddle members to the connector member along the central axis of the paddle member; and flexing each of the paddle members to surround a patient's limb, thereby forming a conical cavity section that is complementary to a conical section of a patient's limb.

One embodiment of the present invention further comprises increasing the flexibility of a paddle member by using a paddle member having a first angle and a second angle that defines the orientation of the wing members in relation to the remainder of the paddle member.

One embodiment of the present invention further comprises increasing the flexibility of a paddle member by using a paddle member with relief cuts.

These aspects of the invention are not meant to be exclusive and other features, aspects, and advantages of the present invention will be readily apparent to those of ordinary skill in the art when read in conjunction with the following description, appended claims, and accompanying drawings.

### BRIEF DESCRIPTION OF THE DRAWINGS

The foregoing and other objects, features, and advantages of the invention will be apparent from the following description of particular embodiments of the invention, as illustrated in the accompanying drawings in which like reference characters refer to the same parts throughout the different views. The drawings are not necessarily to scale, emphasis instead being placed upon illustrating the principles of the invention.

FIG. 1A shows a front view of a prior art one piece paddle.

FIG. 1B shows a side view of a prior art one piece paddle.

FIG. 1C shows a front view of a prior art two piece paddle.

FIG. 1D shows a side view of a prior art two piece paddle.

FIG. 2 shows one embodiment of the support members of an orthotic brace of the present invention.

FIG. 3 shows an anatomical section of a human limb.

FIG. 4 shows one embodiment of the support members of an orthotic brace of the present invention.

FIG. 5A1, FIG. 5A2, FIG. 5A3 and FIG. 5A4 show one embodiment of the paddle member of an orthotic brace of the present invention.

FIG. 5B1 and FIG. 5B2 show one embodiment of the paddle member of an orthotic brace of the present invention.

FIG. 5C1 and FIG. 5C2 show one embodiment of the paddle member of an orthotic brace of the present invention.

FIG. 6 shows a cross-section of one embodiment of the paddle member of an orthotic brace of the present invention on a human limb.

FIG. 7A, FIG. 78 and FIG. 7C show embodiments of the paddle member of an orthotic brace of the present invention.

FIG. 8A shows one embodiment of the paddle member (not flexed) of an orthotic brace of the present invention.

FIG. 8B shows one embodiment of the paddle member (flexed) of an orthotic brace of the present invention.

### DETAILED DESCRIPTION OF THE INVENTION

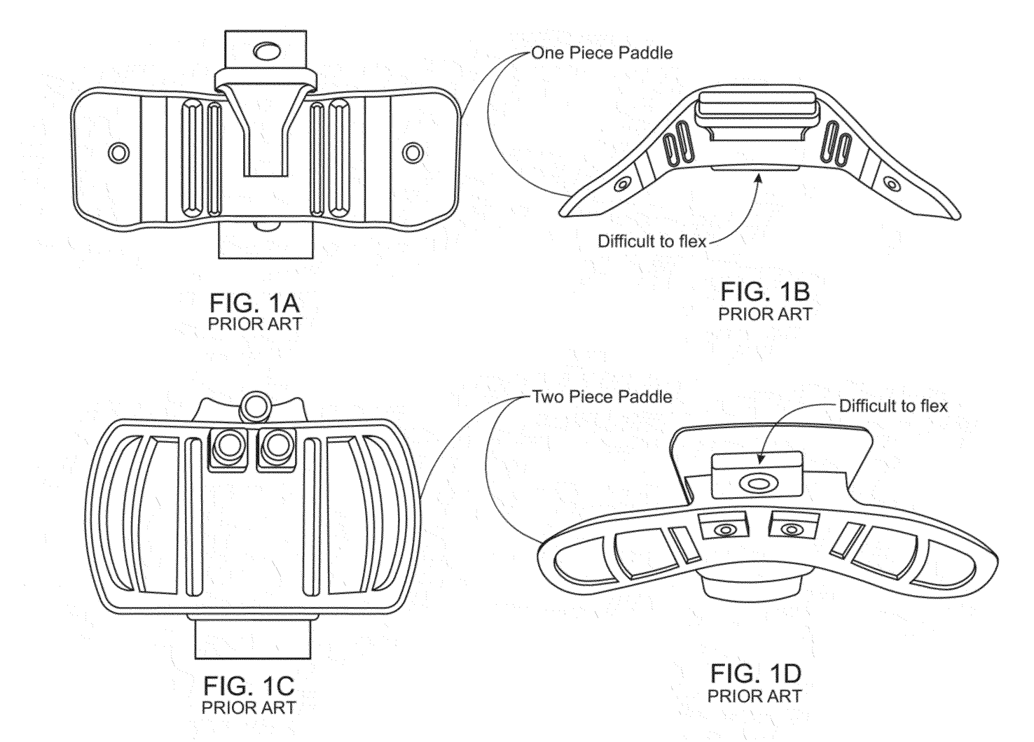

It is typical for a device such as a post-operative knee or elbow brace to attach to a patient by using support members with adjustable straps, cuffs, or pads. In fact, patents as early as U.S. Pat. No. 575,199, issued in 1897 applied these “cuff” concepts. Today, however, brace support members are primarily manufactured in two configurations. The most common method of manufacture is to integrate a paddle member, which fits to the body; and a connector member, which is connected to, and moves along the brace's struts, into one component. See, FIG. 1A and FIG. 1B. The downfall to this design is in the difficulty of finding the right blend of moldable geometry to achieve both flexibility and rigidity at the same time. The combined paddle member/connector member needs to maintain rigid strength around the strut connection and flexible contouring around the patient's leg. These single molded support members are made from one plastic that must achieve very opposite design intents. The result is a mediocre support member with limited conformity.

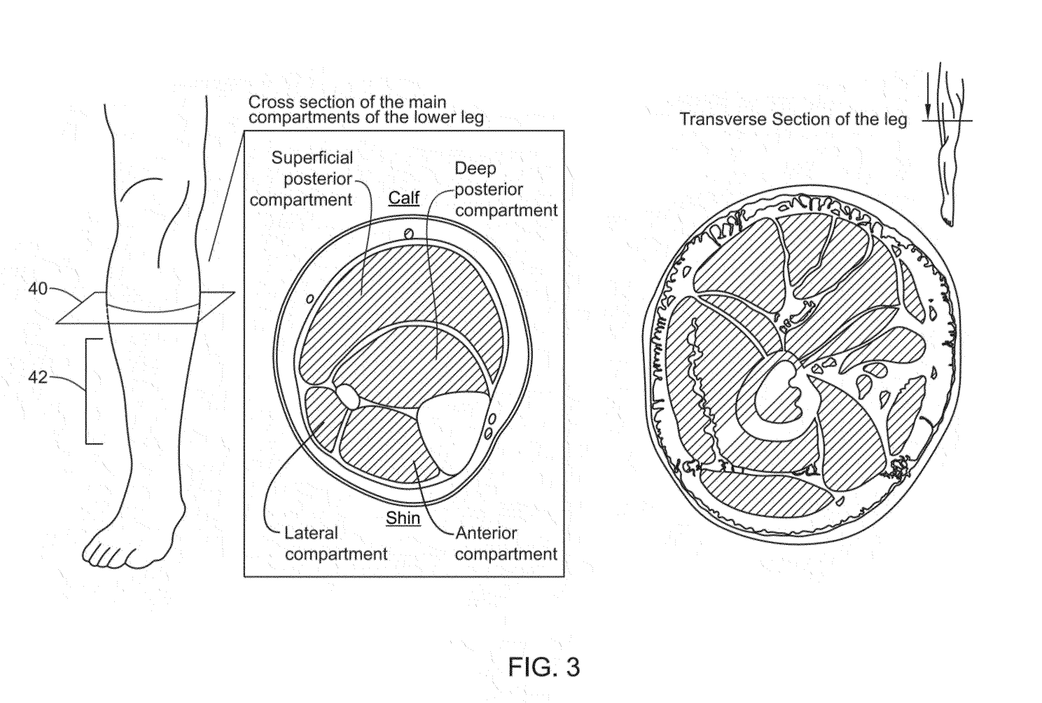

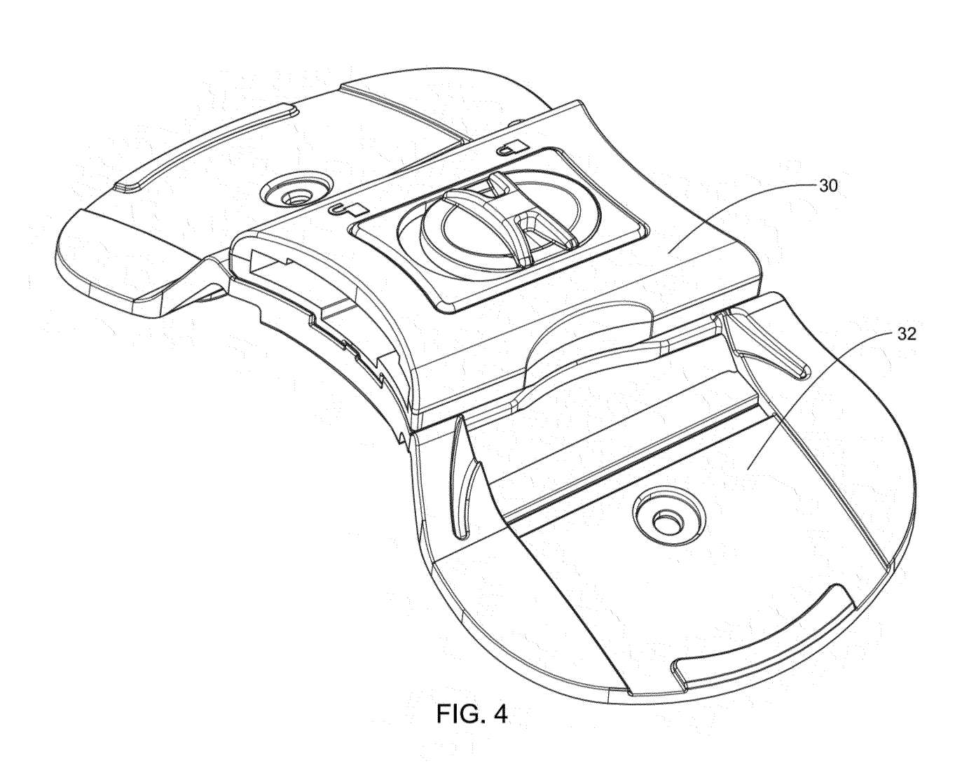

Referring to FIG. 1C and FIG. 1D, the second method of manufacture known in the art is to separate the support member into two distinct parts: a paddle member 2 and a connector member 4. The benefit of this method is the ability to modify the geometry and materials separately. It is common for the connector member 4 to be attached to the actual brace (metal struts) through a channel, tube, or rivet. The connector member 4 becomes the base for the paddle member 2 attachment. Typically, a paddle member 2 has integrated slots for connecting straps that they weave through and are tightened around the user's leg. When the paddle member 2 is tightened around the user's leg, the face 6 of the paddle member forms a cylindrical cavity. The cylindrical cavity does not conform well to the user's leg as the leg consists of conical sections (as described in more detail below). See, for example, FIG. 3.

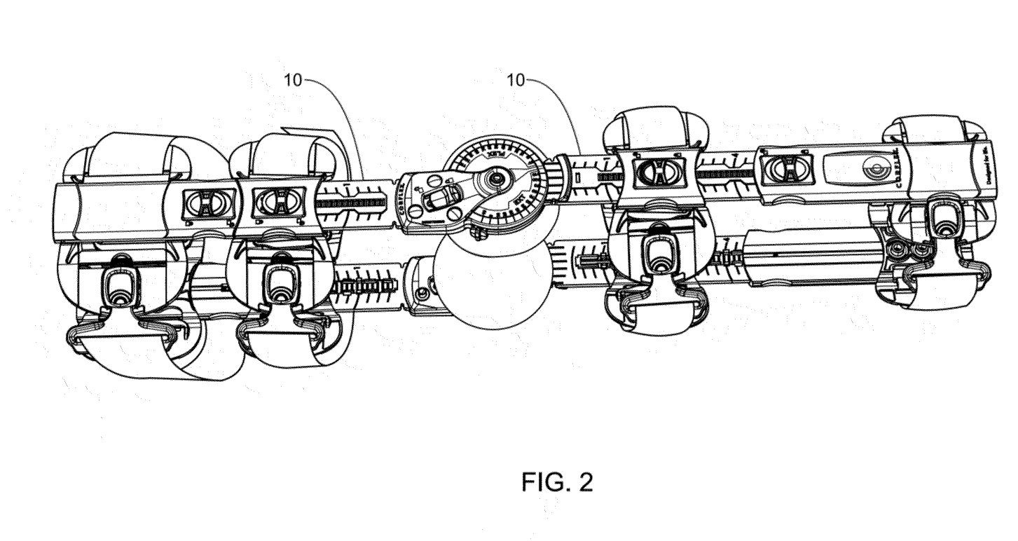

Referring to FIG. 2, one embodiment of the orthotic brace of the present invention that reduces brace migration is shown. One embodiment of the orthotic brace of the present invention comprises two bendable, lightweight struts 10 extending off an adjustable hinge 20 located axially near the knee. One strut extends and telescopes up along the thigh and the other strut extends down the leg along the calf. Located along these struts is a series of support members (designated by arrows).

Attempts have been made to reduce “brace migration” using new paddle Variations, silicone “grip” padding, and even secondary strapping methods. However, considering all the possible variations in this field, none have truly neutralized the problem through paddle geometry until the present invention.

Referring to FIG. 3, an anatomical view of a human limb is shown. A cross-section 40 of the human limb is approximately circular, but a length of the limb 42 is not cylindrical. A length of the 42 limb is generally conical in nature above and below the knee. However, the radius of a cross-section changes inches from a given plane. To properly support a limb, and reduce “brace migration”, the paddle member of the orthotic brace of the present invention takes into account this anatomy.

As shown in FIG. 4, a support member comprises a connector member 30 and a paddle member 32 having a strap (not shown) running radially through it that adjustably connects around the patient's leg. The telescoping struts allow the brace to fit universally to a variety of patients.

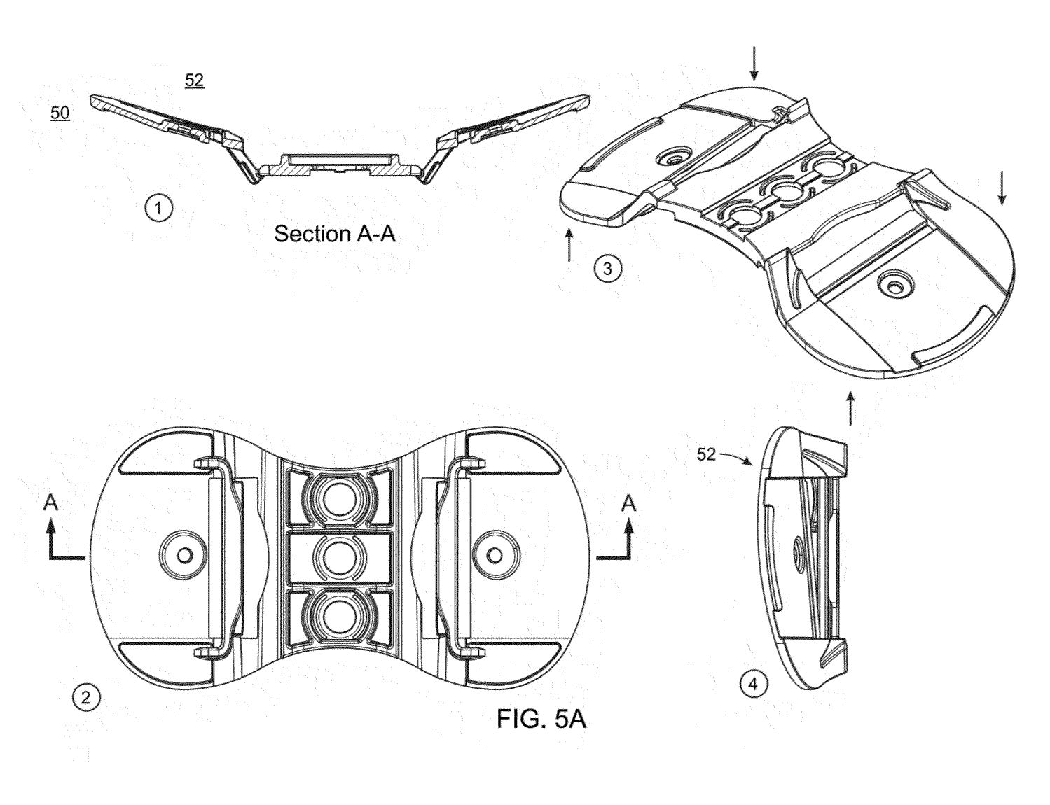

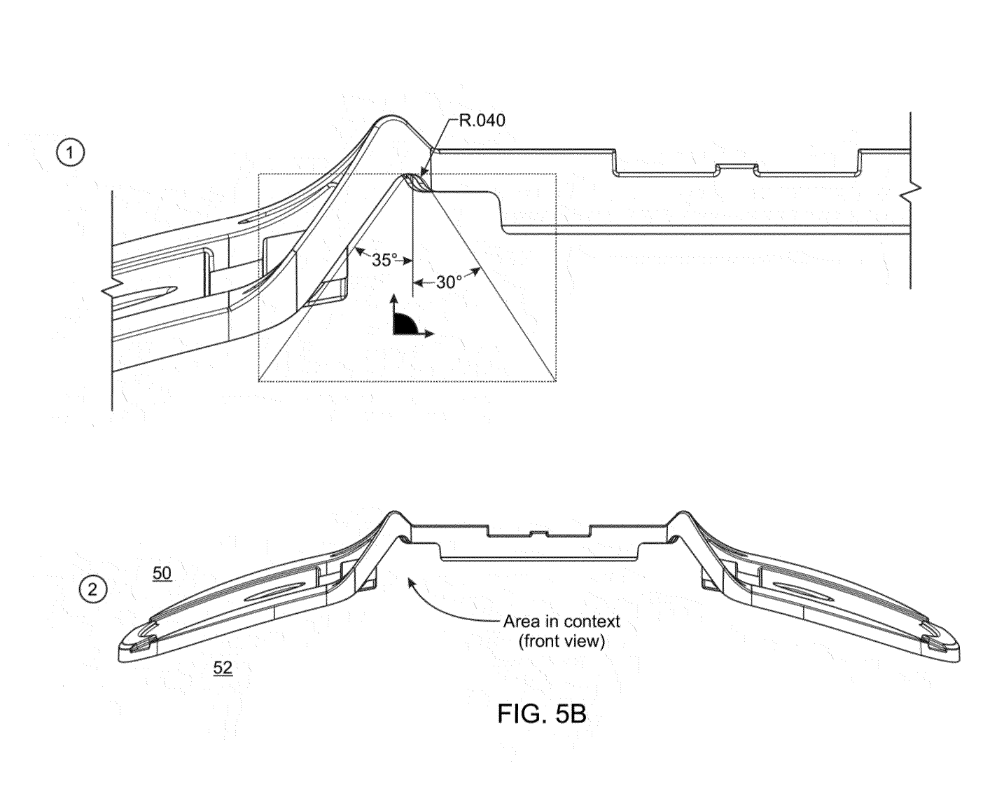

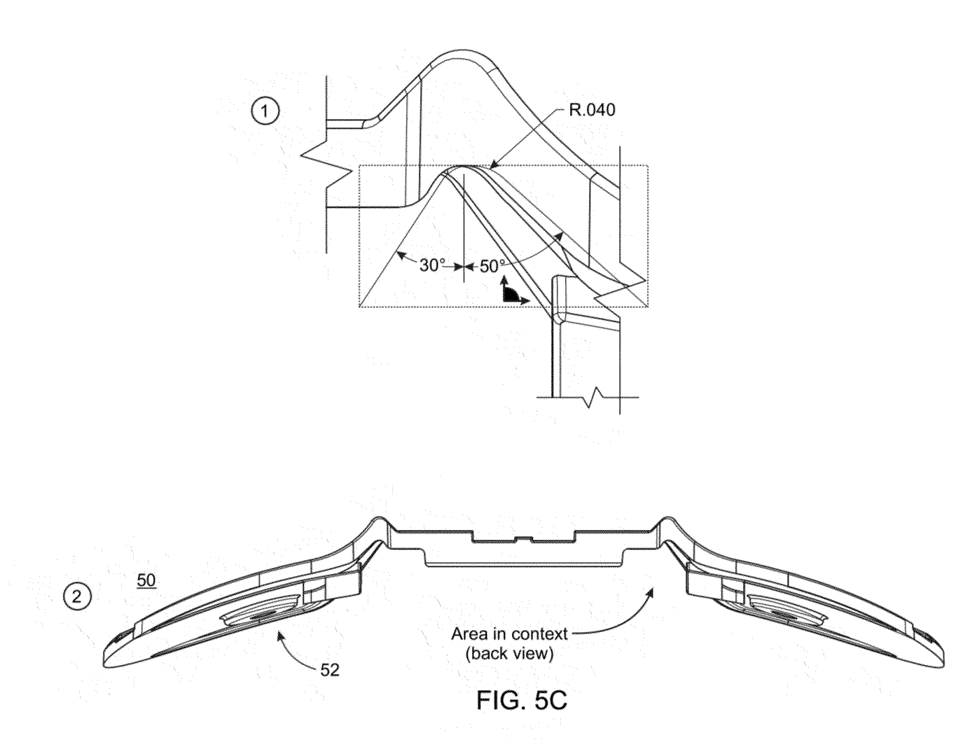

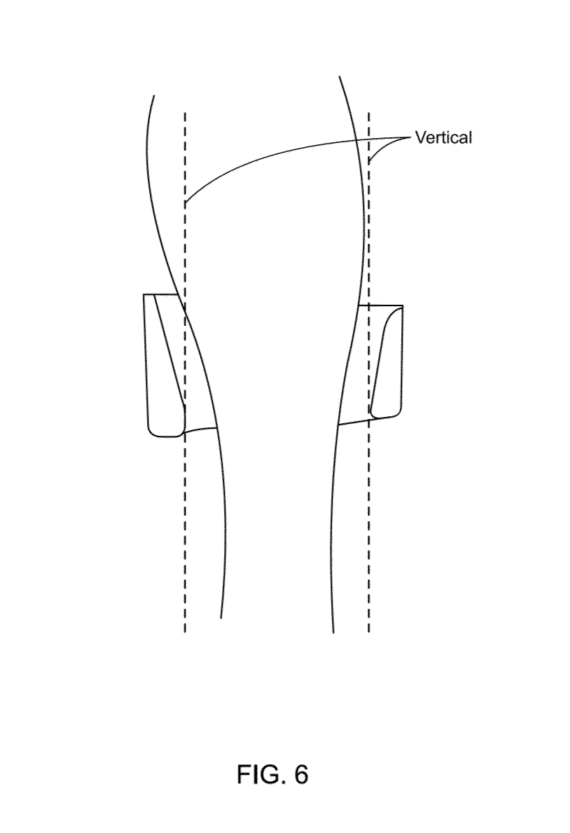

Referring to FIGS. 5A-5C, embodiments of the paddle member 50 of an orthotic brace of the present invention are shown. Referring to FIG. 5A1, the face 52 of the paddle member slopes so that it forms a cavity approximating a conical section when it is flexed around a patient's limb. Referring to FIG. 5A2 and FIG. 5A3, the paddle member is centrally connected to the connector member (not shown) to provide added flexibility and twistability of each side of the paddle member independent of the other side. Here, the central connectivity is shown in the form of three holes, but it is understood by those of skill in the art that several mechanisms are possible.

Referring to FIG. 5A3, the flexibility of the paddle member of the present invention is increased, in part, due to the central connectivity. Here, the ability to flex and/or twist is represented by arrows. It is to be understood that the amount and direction of flexibility can vary from side to side and from front to back (or top to bottom) depending on the location of the paddle member along the strut and to best accommodate the anatomy of a particular user.

Referring to FIG. 5B2 and FIG. 5C2, embodiments of the paddle member of the present invention are shown. In certain embodiments, there are areas of material that are “notched out” to provide enhanced flexibly. Here, some of those areas are shown with arrows. These notches provide a defined flex point that allows the paddle to readily conform to varying geometry of the body.

As shown in FIG. 6, the paddle member forms a conical section that is complementary to the conical section formed by the patient's limb when placed on a patient's limb. The paddle member of the present invention has an improved fit over a paddle member forming a cavity approximating a cylinder as shown in FIGS. 1A-1D.

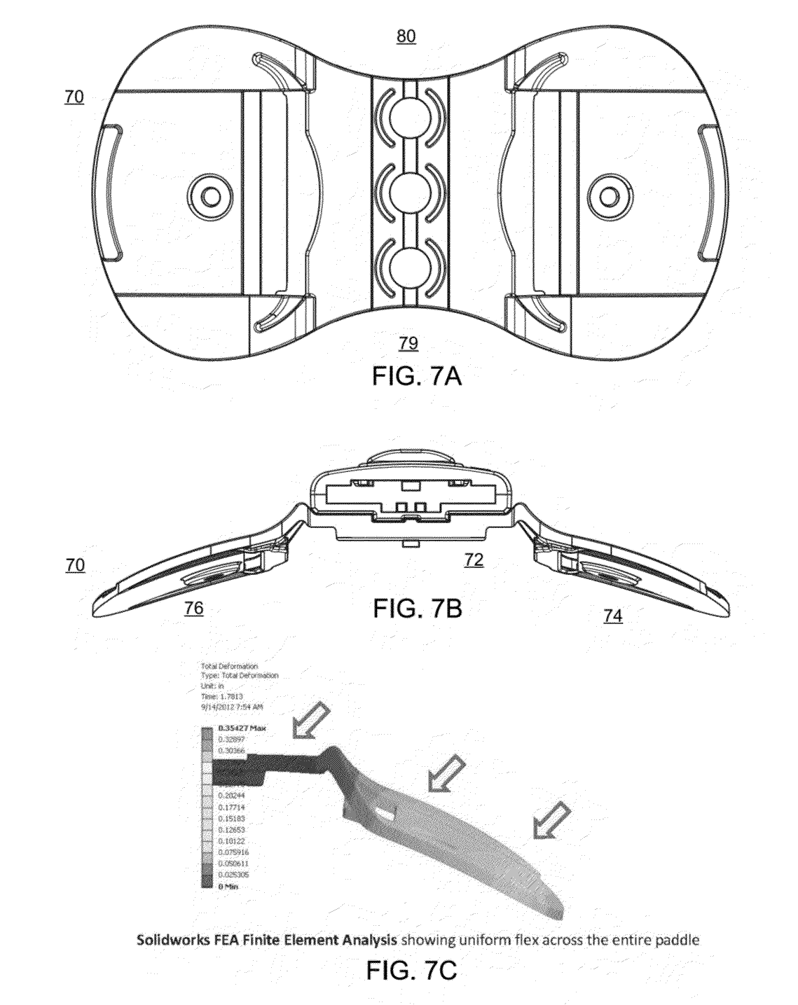

In another embodiment of the present invention, as shown in FIGS. 7A-7C, the paddle member 70 has a center section 72 and wing members 74, 76. As an example, wing member 74 is off-set to center member 72 by two angles 77, 78 each between 90° and 180°. The angles are formed by “notches” or areas lacking material and they provide greater flexibility as shown in FIG. 5B1 and FIG. 5C1. As can be seen in FIG. 5B2 and FIG. 5C2, the angles can vary from front to back depending on the desired geometry of the paddle member. In certain embodiments, the two angles are the same.

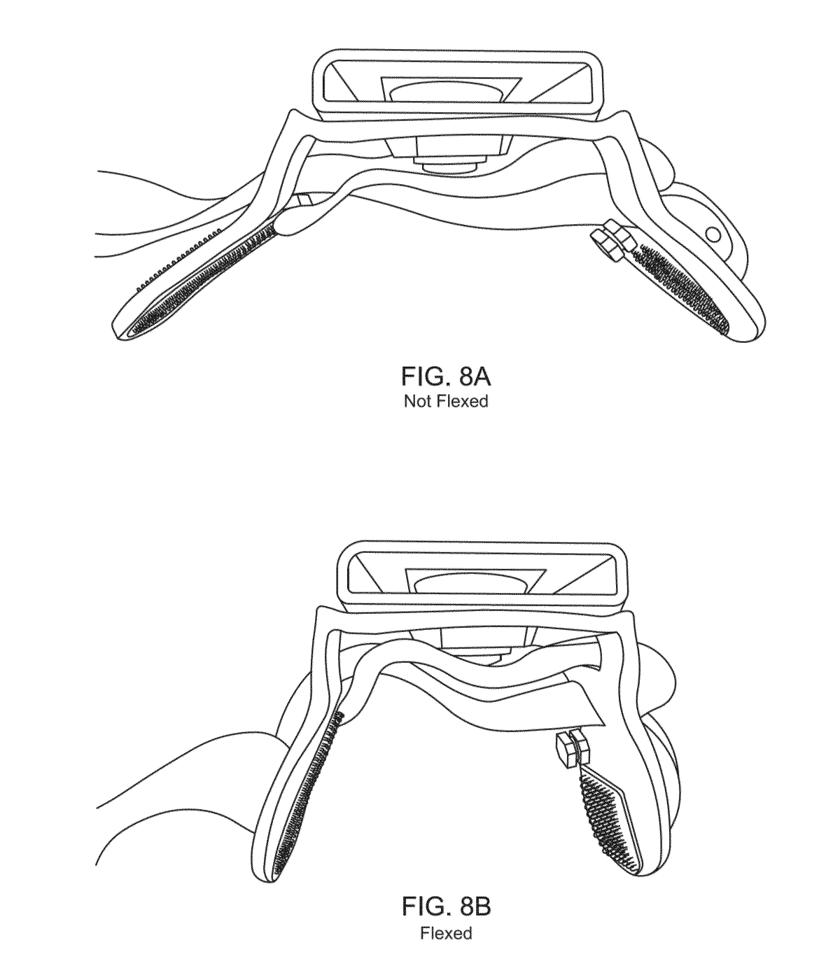

Still referring to FIGS. 7A-7C, in another embodiment of the present invention the paddle member 70 has relief cuts 79, 80. The offset wing members and the relief cut allow the paddle member to be flexed more easily and to better fit the contour of the user's limb, as shown in FIGS. 8A-8B.

Referring to FIG. 8A and FIG. 8B, one embodiment of the paddle member of the present invention is shown. In FIG. 8A, the paddle member is “not flexed,” but does show sloping wing members and angles that provide for increased flexibility and conformability. In FIG. 8B, the paddle member is “flexed” and shows how the faces of the wing members provide a conical, conformable cavity that can match the limb of a patient with greater accuracy and comfort. The ability to better conform to a patient greatly reduces brace migration and increase compliance in a patient due to increased comfort and fit. Both of these features aid in patient healing.

The preferred method of making the paddle member of the present invention focuses on the geometry and modular nature of the method of manufacture. The support member assembly utilizes a multi-piece or modular methodology. In one embodiment, the connector member comprises an injection molded plastic. In certain embodiments, the plastic comprises ABS, Nylon, Polyester, polycarbonate, polysulfons, polyphenylene sulphide, polystyrene, PVC, Styrene, Acrylonitrile, and compounded resins. The selected material properties must 1) be rigid enough to stay affixed to the metal struts, 2) potentially accept a button mechanism, and 3) not fracture under stress. In one embodiment, the paddle member comprises an injection molded plastic. In certain embodiments, the plastic comprises polypropylene, polyethylene, polybutene, nylon, TPE, compounded resins, and the like. It is important that the paddle member flex repeatedly without physically breaking down or distorting.

The benefits of this invention include a better fit of the brace to the leg, minimized brace migration and increased patient comfort. These benefits help alleviate one of the greatest problems with rehabilitative braces in general: non-compliance to wear the brace. The modular construction offers a range of flexible materials for the paddle members to be molded. The value added from the properly specified plastic allows the flexible paddle member to conform as it changes shape through increased movement.

While the principles of the invention have been described herein, it is to be understood by those skilled in the art that this description is made only by way of example and not as a limitation as to the scope of the invention. Other embodiments are contemplated within the scope of the present invention in addition to the exemplary embodiments shown and described herein. Modifications and substitutions by one of ordinary skill in the art are considered to be within the scope of the present invention.

What is claimed:

1. An adjustable orthotic brace, comprising:
at least one telescoping strut; and
a plurality of support members located along the telescoping strut, wherein each of the support members has a connector member and a paddle member, wherein the paddle member is centrally connected to the connector member and has a pair of wing members with faces that form a conical cavity section when the paddle member is flexed to surround a patient's limb.

2. The adjustable orthotic brace of claim 1, wherein the conical cavity section formed by the faces of the pair of wing members is complementary to a conical section of a patient's limb.

3. The adjustable orthotic brace of claim 1, wherein each of the paddle members further comprises a first angle and a second angle that define the orientation of the wing members in relation to the remainder of the paddle member allowing greater flexibility of the paddle member.

4. The adjustable orthotic brace of claim 1, wherein each of the paddle members further comprises relief cuts that allow the paddle member to flex more easily to conform better to a patient's limb.

5. A method for reducing orthotic brace migration, comprising:
providing an adjustable orthotic brace with at least one telescoping strut;
providing a plurality of support members located along the telescoping strut, wherein each of the support members has a connector member and a paddle member and the paddle member has a central axis and a pair of wing members;
connecting each of the paddle members to the connector member along the central axis of the paddle member; and
flexing each of the paddle members to surround a patient's limb, thereby forming a conical cavity section that is complementary to a conical section of a patient's limb.

6. The method for reducing orthotic brace migration of claim 5, further comprising increasing the flexibility of a paddle member by using a paddle member having a first angle and a second angle that defines the orientation of the wing members in relation to the remainder of the paddle member.

7. The method for reducing orthotic brace migration of claim 5, further comprising increasing the flexibility of a paddle member by using a paddle member with relief cuts.
